(19) United States
(12) Patent Application Publication
(10) Pub. No.: US 2020/0206379 A1
(43) Pub. Date: Jul. 2, 2020

(54) Enclosed Space Air Treating Assembly

(71) Applicant: Jeffrey Dean Heyd, Palm Desert, CA (US)

(72) Inventor: Jeffrey Dean Heyd, Palm Desert, CA (US)

(21) Appl. No.: 16/720,078

(22) Filed: Dec. 19, 2019

Related U.S. Application Data
(60) Provisional application No. 62/786,509, filed on Dec. 30, 2018.

(51) Int. Cl. A61L 9/14; A61L 2/16; A61L 2/24

(52) U.S. Cl. CPC A61L 9/14; F24F 7/06; A61L 2/24; A61L 2/16

(57) ABSTRACT

An enclosed space air treating assembly includes a compartment having an interior. The compartment has an air inlet and an air outlet, wherein pressurized air enters into the interior through the air inlet and exits the interior through the air outlet. Pressurized air received by the air inlet is defined as received air. An atomizer is in fluid communication with the interior and supplies atomized fluid to the received air within the interior such that that the atomized fluid is carried outwardly through the air outlet. A control is in communication with the atomizer. The control turns on the atomizer intermittently for a preselected amount of time while the pressurized air flows through the compartment.

### CROSS-REFERENCE TO RELATED APPLICATIONS

I hereby claim the benefit under 35 U.S.C. Section 119(e) of U.S. Provisional application 62/786,509 filed on Dec. 30, 2018.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to enclosed wall space treating and drying systems and more particularly pertains to a new enclosed wall space treating and drying system for allowing intermittent addition of atomized fluids to the air flow of wall space treatments.

Frequently enclosed spaces in structures such as homes, apartments, offices, industrial buildings, office buildings and the like are dampened, wetted, soaked, flooded (collectively “wetted” herein) by water. An enclosed space subject to water intrusion is normally a system of spaces, such as the enclosed spaces behind a wall where spaces are separated by studs. It should be understood that such enclosed spaces may include spaces within walls, cabinetry, roofing and flooring. Such water intrusion can be caused by (1) water overflow from bathroom basin, sink, shower, bathtub, toilet, dishwasher, or washing machines, (2) snow and/or rain leakage through roofs or walls, (3) plumbing leakage, (4) flooding by heavy rains, river overflows, ocean wave action, tidal actions, and the like.

Wetted enclosed spaces often create a favorable environment for microbes and mold growth especially if the water source was previously contaminated such as in the case of overland flooding or sewage backflows. Microbe and mold growth can lead to unpleasant aromas and/or health issues. Molds can cause pulmonary infections and such infections can be life threatening especially for infants, young children, the sickly and the elderly.

Microbe and mold growth normally will start within 24 to 48 hours after wetting. Once mold growth starts, mold spores are created. When an enclosed space is dried after wetting, the microbes may die, but some microbes form spores, and molds may die but the mold spores are not necessarily killed and can remain a health hazard in any structure with human or animal inhabitants.

Classically when enclosed spaces in structure were wetted, the enclosed space was open to the air to permit thorough drying. This was typically accomplished by removing at least one wall or covering to open the enclosed space to the open air and to permit free air exposure to the space and all surfaces in the space. Thus, if the enclosed space was a wall in a home, one of the walls of the enclosed space was removed to expose the space and all surfaces in the space. The removed wall could be a plaster wall, a plaster board wall, a wood plank wall, a plywood wall, and the like. It was a major undertaking and expensive since the wall had to be replaced once the exposed surfaces of the previously enclosed space were dried.

In recent years, the approach has been to inject an air stream, i.e. forced air, into the enclosed space to dry the exposed surfaces. Depending upon the humidity of the forced air, the air temperature and the degree of wetness in the enclosed space, the drying operation could take from one to seven days on the average. Longer periods are sometimes necessary.

The challenging thing about recovering from water damage is that you can typically only see a small part of the actual damage. The majority of the affected space and surfaces exposed in the enclosed space is often hidden, such as the enclosed space behind the walls. It's critical to identify and dry all of the affected areas to prevent kill the microbes and mold. But if the enclosed space is wetted again, the microbe spores and mold regenerate the microbes and mold.

The methods for dealing with damage to walls subject to wetting depend on the type of materials and also what's behind those materials. Drywall can often be salvaged if the drying operation is promptly carried out. The drying approach will depend upon contents in the enclosed space, that is, the space behind the wall. If the enclosed space has insulation, the enclosed space is usually reached with flood cuts through a wall of the enclosed space. If there is no insulation in the enclosed space, weep holes are usually created in an enclosed space wall. If the enclosed space has a firewall, staggered cuts are usually made in the firewall to reach the enclosed space. Even with the present force air drying methods, sometimes removal of one wall of the wetted enclosed space must be carried out to dry the wetted area.

Although forced air stream drying is usually quite effective for drying the surfaces of the enclosed space, it does not usually eliminate the microbe and mold spores that have formed from the wetting. If the ambient air has period of high humidity, and during periods of normal rain, some of the microbe and mold spores remaining in the enclosed space after the forced air drying treatment can be reactivated and can cause unpleasant aromas and/or create a health hazard from the reactivated mold.

Enclosed spaces, typically the spaces in the wall and ceilings of structures, are not sealed and are not air tight. In a typical building the walls are comprised of a series of studs supporting walls, such as walls constructed of plaster board. Because of holes in the studs, or porous knots in the studs or pacing between the stud and plater board, the enclosed spaces between the plaster board and studs are not air tight. Air can be blown in the enclosed space because there are many exit avenues for the forced air in the space. Similarly, air can be pulled or drawn out of such enclosed spaces by a vacuum draw because there are many inlet avenues into the space from other areas and enclosed spaces that permit air to be drawn into the enclosed space from such other areas or enclosed spaces. This permits the operation of push-pull. The air blower for the drying is used to force drying air into the enclosed space and the vacuum side of the air blower, the pull side, is used to draw the spent forced air from the enclosed space.

Although the conventional drying equipment and techniques are effective for drying enclosed spaces subject to wetting, they do not sanitize the enclosed space and the dried enclosed space frequently harbors microbe and mold spores after the drying operation.

What is required is an effective way of eliminating microbes and molds and microbe and mold spores from a wetted enclosed space before, during or following the drying operation. In some instances the microbes and molds can be more easily eliminated before the drying, while in others the microbes and molds can be effectively eliminated during the drying operation. Sometimes the enclosed space must be treated after the drying operation to eliminate the microbe and mold spores.

Ideally what is needed is a way to dry an enclosed space and simultaneously kill, treat, and/or eliminate mold and mold spores. An effective way to treat a wetted enclosed space would be to treat the space with a chemical agent that can counteract odors, kill microbes and/molds and/or kill microbe and/or mold spores. Such agents include pesticides, sanitizers, fungistatics and/or fungicides. Some agents work best in wet environments, others work best in dry environments. The treatment must thusly be tailored for the particular problem.

It would be desirable also to be able to treat an enclosed space during the drying operation with an odor-counteractant agent to prevent the formation of and to eliminate unpleasant orders arising from the wetting of the enclosed space. It would be desirable also to be able to treat an enclosed space as part of or during the drying operation with a sanitizing agent to kill and eliminate microbes, to prevent microbial growth and to molds and to prevent mold growth. The treatment as mentioned above might have to be done in stages before, during and after the drying operation. But it would be advantageous to have everything set up so that treatment and drying can be carried out with the same gear already set up.

It would also be desirable to be able to treat an enclosed space during the drying operation with an insecticide to eliminate and/or kill insects. Some chemical agents can be effectively used when evaporated into the forced air used for drying in the gaseous state. But most chemical agents are more effectively used as a fog or mist of fine droplets/particles that can adhere to coat the exposed surface of the enclosed spaced be treated after being wetted.

### (2) Description of Related Art Including Information Disclosed Under 37 CFR 1.97 and 1.98

The prior art relates to enclosed wall space treating and drying systems. Such systems typically are used to blow ambient air into enclosed wall spaces to dry any wetted surfaces. However, such devices do not effectively treat detrimental conditions within the enclosed spaces such as mold and fungus growth, insects, and odors which can permeate the walls. Moreover, such devices often require an entire room to be heated such that the ambient hair is heated air that enters the blower. This reduces the useful life of the blower while creating uncomfortable conditions within the room.

### BRIEF SUMMARY OF THE INVENTION

An embodiment of the disclosure meets the needs presented above by generally comprising a compartment having an interior. The compartment has an air inlet and an air outlet, wherein pressurized air enters into the interior through the air inlet and exits the interior through the air outlet. Pressurized air received by the air inlet is defined as received air. An atomizer is in fluid communication with the interior and supplies atomized fluid to the received air within the interior such that that the atomized fluid is carried outwardly through the air outlet. A control is in communication with the atomizer. The control turns on the atomizer intermittently for a preselected amount of time while the pressurized air flows through the compartment.

In one embodiment the atomizer may comprise a sonic transducer while in another it may comprise a nozzle capable of creating a mist when supplied with fluid under pressure. The fluid may include any suitable fluid or chemical agent utilized for controlling odors, preventing the growth of mold, eradication of insects, destroying bacteria and the like. The fluid may also include combinations of the above though co-mixing may not be available for all fluids and separate treatments may be required. Separate atomizers may be utilized or separate reservoirs of different fluids may be provided.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a side view of a enclosed space air treating assembly according to an embodiment of the disclosure.

FIG. 2 is a top view of an embodiment of the disclosure.

FIG. 3 is a side, cross-sectional view of an embodiment of the disclosure.

FIG. 4 is a cross-sectional view of an embodiment of the disclosure taken along line 4-4 of FIG. 3.

FIG. 5 is a side view of an embodiment of the disclosure.

FIG. 6 is a top view of a control of an embodiment of the disclosure.

FIG. 7 is a schematic view of an embodiment of the disclosure.

FIG. 8 is a top view of an embodiment of the disclosure.

FIG. 9 is a side view of an embodiment of the disclosure.

FIG. 10 is a side view of an embodiment of the disclosure.

FIG. 11 is a schematic view of an embodiment of the disclosure.

### DETAILED DESCRIPTION OF THE INVENTION

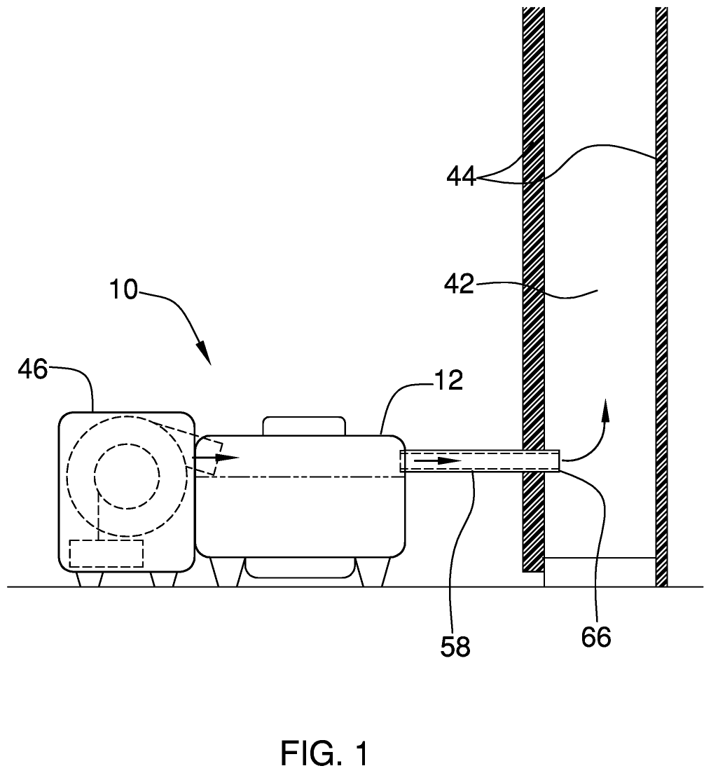

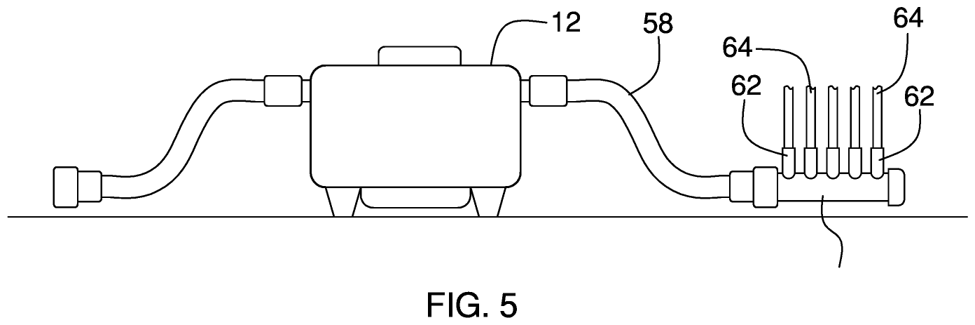

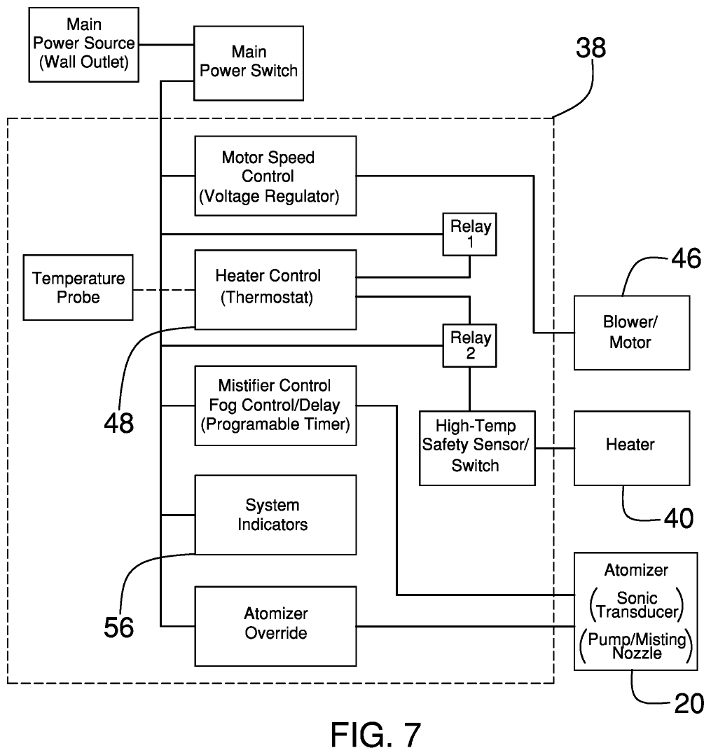

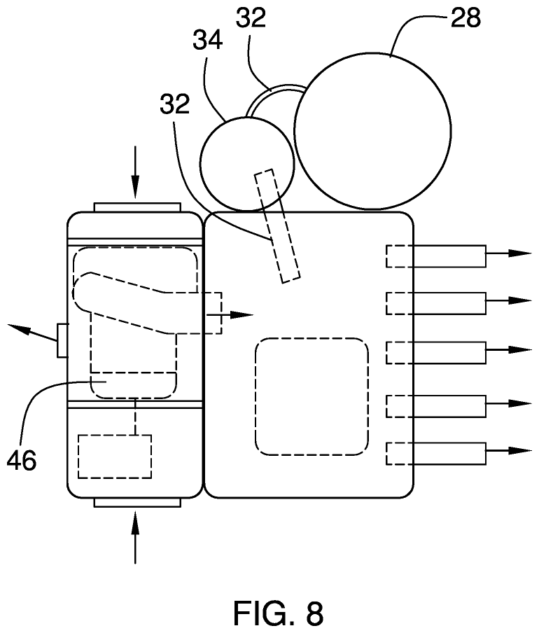

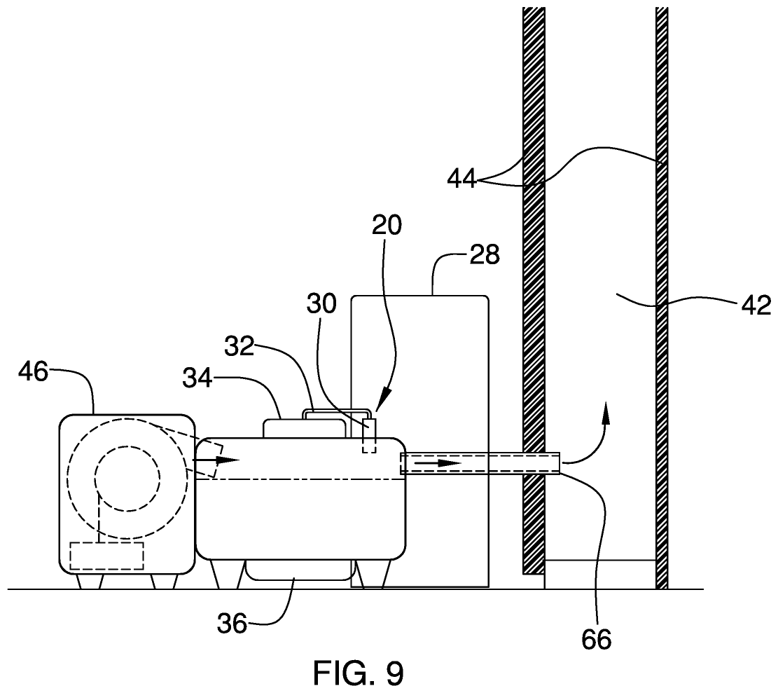

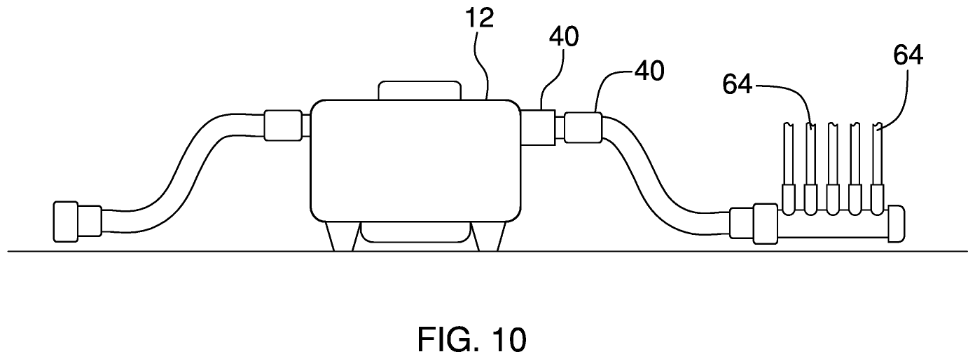

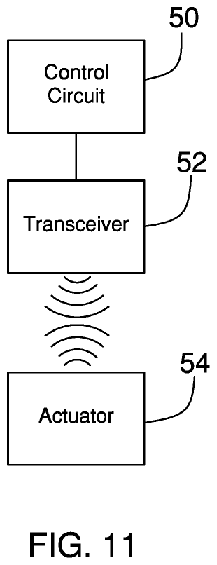

With reference now to the drawings, and in particular to FIGS. 1 through 11 thereof, a new enclosed wall space treating and drying system embodying the principles and concepts of an embodiment of the disclosure and generally designated by the reference numeral 10 will be described. It should be understood that the Figures will show various combinations of elements of the enclosed space air treating assembly 10. Thus, while some Figures may include particular elements, others will not and therefore while not all possible combinations of the elements will be displayed in the Figures, it is understood that any combination of the elements discussed below is contemplated by the current disclosure.

As best illustrated in FIGS. 1 through 11, the enclosed space air treating assembly 10 generally comprises a compartment 12 having an interior 14. The compartment 12 has an air inlet 16 and an air outlet 18. Generally, pressurized air is directed to and enters into the interior 14 through the air inlet 16 and exits the interior 14 through the air outlet 18. For the purpose of clarification and to facilitate the function of the assembly 10, pressurized air received by the air inlet 16 will be defined as received air. The source of the pressurized air will be described in more detail below.

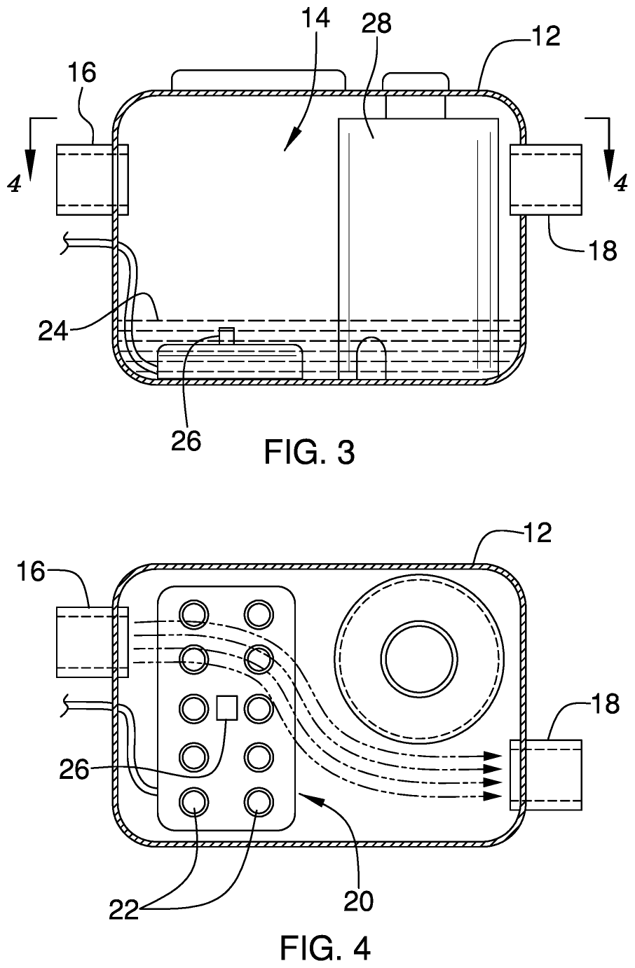

An atomizer 20 is in fluid communication with the interior 14 and is configured to supply atomized fluid to the received air within the interior 14 such that that the atomized fluid is carried outwardly through the air outlet 18. The term atomized or atomization as used herein with respect to fluids should be considered analogous to those devices which create droplets of fluid between 0.1 microns and 30 microns in size. Consequently, atomization may be replaced by misting, fogging, nebulization, and the like, each of which differ from evaporation of the fluid. In one embodiment, the atomizer 20 comprises a sonic transducer 22 configured to atomize a fluid in contact with the sonic transducer 22. The term “sonic transducer,” as used herein, is used to define convention sonic and ultrasonic transducers which, when turned on while in contact with a fluid 24, are capable of atomizing the fluid 24. In an embodiment as shown in FIG. 3, the interior 14 is configured to hold a quantity of fluid 24 and the sonic transducer 22 is positioned such that the sonic transducer 22 is in contact with the fluid 24. Typically a plurality of sonic transducers 22 will be used simultaneously and will vibrate at an extremely high frequency, such as, for example, 1.65 million vibrations per second. A fluid level detector 26 may be provided for turning of the sonic transducer 22 should the fluid level drop below a point where the sonic transducer 22 is no longer submerged so that the sonic transducer 22 is turned off to prevent overheating.

While the compartment 12 may contain the fluid 24 in such a quantity that the compartment 12 itself is regularly filled with the fluid 12, a reservoir 28 may be added which is in fluid communication with the compartment 12 such that the fluid 24 in the reservoir 28 flows into the interior 14 as needed. This may be accomplished with a mechanical float and valve combination apparatus controlling gravity fed fluid to the interior 14 or a pump may be utilized to pump the fluid 24 from the reservoir 28 to the interior 14. Regardless, the reservoir 28 may be filled as needed without having access to the interior 14. The reservoir 28 may be formed as a unitary structure attached to or placed within the compartment 12 or may be a separate structure.

In another embodiment, the atomizer 20 may comprise a nozzle 30, or mistifier nozzle, through which the fluid 24 is ejected under very high pressures such as between 1000 psi and 3000 psi but this may be lowered should the fluid be mixed with air before being ejected by the nozzle 30. The reservoir 28 holds a quantity of fluid 24 and a conduit 32 fluidly connects the nozzle 30 with the reservoir 28 such that the conduit 32 delivers the fluid 24 to the nozzle 30. It should be appreciated that as with the sonic transducer 22 embodiment, the interior 14 of the compartment 12 itself may serve as the reservoir since the pressurized air may simply flow over the fluid 24 when it is in a non-atomized state. Further, the reservoir 28 may again be either attached to the compartment 12 or provided as a separate structure as shown in FIG. 8. A pump 34 is in fluid communication with the conduit 32. The pump 34 pumps the fluid from the reservoir 28 to the nozzle 30 when the pump 34 is turned on with a sufficient enough pressure to cause the fluid 24 to be atomized into a mist that will be directed into the pressurized air. In this embodiment, the interior 14 may simply comprise an air conduit as opposed to a housing type structure shown in the Figures. The interior 14 may include a condensate collector 36 to capture any condensation formed during usage of the nozzle 30.

A control 38 is in communication with the atomizer 20. The control 38 turns on the atomizer 20 intermittently for a preselected amount of time while the pressurized air flows through the compartment 12. That is, while the pressurized air may flow through the compartment 12 for an extended amount of time, the predetermined time constitutes a fractional portion of that extended time. While the atomization may occur at any time, the preselected amount of time will typically occur only when the pressurized air flows through the compartment 12. Should the pump 34 be used with a nozzle 30, the pump 34 will be in communication with the control 38 such that the pump 38 only supplies fluid to the nozzle 30 at times set generally by the parameters herein or by the user of the assembly 10.

The assembly 10 may be operated under several varying combinations of conditions, some of which will be described herein for illustrative purposes. A first condition is defined wherein the atomizer 20 is turned off and no pressurized air flows through the interior 14. The first condition generally includes the assembly 10 not in operation. A second condition is defined wherein the pressurized air flows through the interior and the atomizer 20 is turned off. The second condition, for reasons described below, will typically comprise a vast majority of the operating usage of the assembly 10. A third condition is defined wherein the pressurized air flows through the interior and the atomizer 20 is turned on. When the atomizer 20 is to be used, the second condition is typically maintained by the control for at least 50.0 minutes out of each hour that the pressurized air flows through the interior, whereas the third condition is typically maintained by the control for at least 10.0 seconds out of each hour that the pressurized air flows through the interior. Generally 50.0 minutes would be an absolute minimum for the first condition and it would be unusual for the second condition to be utilized for less than 55.0 minutes out of each hour. For reasons described below, the third condition will preferably be maintained for less than 5.0 minutes per 1.0 hour of operation of the assembly 10, and more preferably 2.0 minutes or less. Additionally, there may be some applications where the third condition is maintained for 30 seconds or less out of each hour the assembly 10 is producing air to be injected into a wall space. However, it is the third condition which will typically dictate the parameters as different fluids 24 will require different application times. However, as mentioned above, the third condition will nearly always utilize 1/12 or less of the time of assembly 10 operation.

It should be understood, that terms hours, minutes and seconds are being used as examples only and that the intermittent nature of the atomizer being operated may be determined by any “units” of time as long as the fraction of time that the atomizer 20 is operational compared to the time pressurized air moves through the interior is within the parameters stated above. Thus, the third condition could be maintained, for example, for 5 minutes out of every 100 minutes that the interior 14 receives air. However, since it is conventional to use drying devices for a set number of hours and the atomizer 20 will typically be used for less than 2 to 3 minutes per hour, the “hour” having been selected as the time unit for explanation purposes.

A heater 40 is in fluid communication with the interior 14 and is configured to heat the received air. Alternatively, the heater 14 may be positioned to heat the pressurized air after it has exited the interior and before it is supplied to the interior wall space 42 of a wall 44. Generally, the heater 40 will heat any pressurized air after the air is pressurized by a blower 46. This positioning is utilized to reduce wear on a blower motor of a blower 46 that is fluidly coupled to the air inlet 16. The blower 46 delivers the pressurized air to the air inlet 16 when the blower 46 is turned on. However, supplying heated air to a blower 46 reduces the lifespan of the blower 46 as its components would be subjected to air that is often heated in excess of 120° F. While the blower 46 is shown in FIG. 1 as a separate unit, the compartment 12 may be divided into chambers such that the blower 46 is positioned within the compartment in one chamber while the atomizer 20 is placed in a separate chamber.

The heater 40 is in communication with the control 38 wherein the control 38 turns the heater 40 on during the second condition. Typically, the control 38 will turn the heater 40 off during the third condition and the heater 40 will be turned off when the blower 46 is turned off. This will prevent the atomized fluid from evaporating as it travels to the wall space 42. It is important that the fluid 24 remain in an atomized state, and not a gaseous state, so that it can attach itself to the surfaces bounding the interior, or enclosed, wall space 42. Therefore, the control 38 will not only intermittently control the atomization of the fluid 24, but also intermittently turn on the heater 40 only during those times of atomization where the evaporation of the fluid 24 into a gas is not preferred. The heater 40 may include any conventional means utilized for heating air though electric heating coils would typically be utilized. However, a propane or other gas heater may also be incorporated into the assembly 10. A thermostat 48 may be in communication with the flow of air between the heater 40 and the wall space 42 and in communication with the heater 40 to control the temperature of the flow of air.

The blower 46 may include multiple other settings such as at least a high output and a low output. The blower 46 may be in communication with and actuated by the control 38 wherein the control 38 turns on the blower 46 at the high output during the second condition but turns on the blower 46 at the low output during the third condition. This is advantageous as at high output back pressure from air moving into the wall spaces 42 will hinder movement of the atomized fluid into the wall spaces 42. A lower air speed, and thus lower air pressure with reduced back pressure, has been found to more effectively move atomized fluid through the wall spaces 42. The air may be treated before entering the blower 46 by an air filter which is used to capture any particulate found in the air being moved by the blower. Additionally, a water trap may be in communication with the blower 46 to prevent water particles from entering and damaging the blower 46. If the air being moved by the blower 46 is high in humidity, the air may be treated with a dryer such as for example with a desiccant dryer.

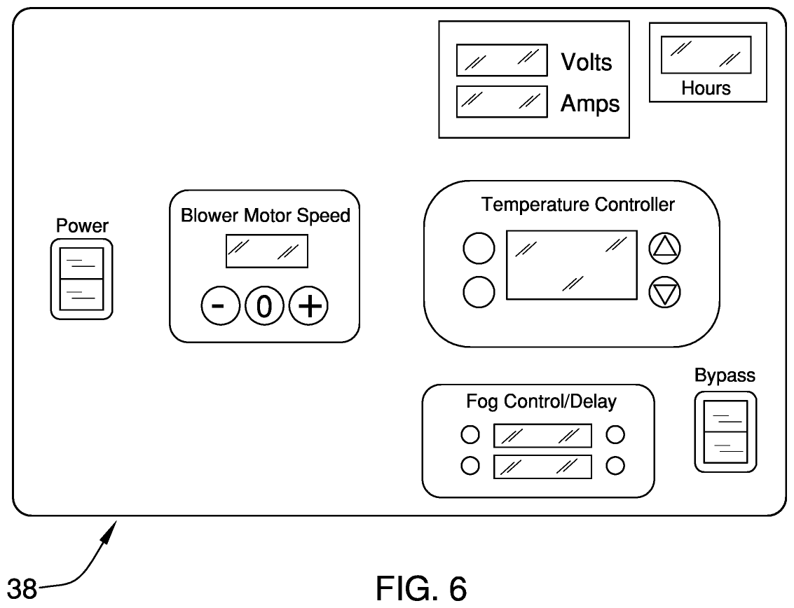

The control 38 may be provided in any number of conventional structures. For example, the control may include a keypad, such as the one depicted in FIG. 6, mounted on the compartment 12 and including input keys for selecting the amount of time the atomizer 20 will operate relative to time the blower 46 is being used. The control 38 will typically include a processor, control circuit 50 or the like programmed to set the blower 46 and heater 40 settings according to the type of fluid 24 being used and the purpose for which the assembly 10 is being used. Thus, the control 38 may include pre-programmed settings for different fluids such as odor-counteractants, sanitizers for reducing microbial growth, fungistatics to inhibit mold proliferation, fungicides, or pesticides. Each fluid 24 will typically have its own preferred settings as its ability to remain atomized and not transition to a gas will differ from fluid to fluid. Additionally, each fluid 24 will have its own effective dispersive volume wherein some fluid will require less than one minute per hour for an effective coverage while others may require multiple minutes per hour. Consequently, the control 38 may be programmed for each fluid 24 anticipated to be used with the assembly 10. Furthermore, the control 38 may be programmed to accept input as to the total volume of interior wall space which will be treated with the atomized fluid. Thus, the control 38 will increase or decrease the amount of time that fluid is being atomized.

In one embodiment, the control 38 includes a transceiver 52 that is configured to be in wireless communication with an actuator 54. Thus, the control 38 may be actuated wirelessly and remotely by way of Wi-Fi, Bluetooth, cellular or other radio frequencies, for example. The actuator 54 may include a stand-alone actuator in communication with the control 38 or may comprise an application which may be utilized in combination with a personal electronic device such as a cellular phone, computer, or tablet computer, for example. The assembly 10 may include conventional system indicators 56 for humidity, temperature and fluid levels which may be transmitted to the actuator 56 for viewing by an operator of the assembly 10.

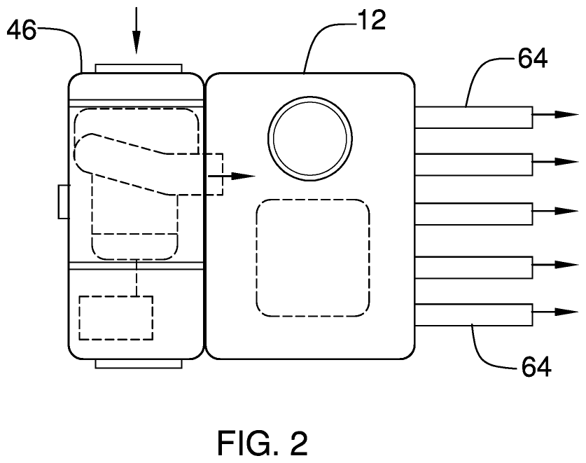

In use, the air outlet 18 is fluidly coupled to delivery network of tubing 58 or conduits which, in turn, carries the pressurized air to the interior wall spaces 42 requiring treatments and/or drying. This may be facilitated by usage of a manifold 60 that is directly coupled to the air outlet 18, or directly to the compartment 12 as shown in FIG. 2, and which includes a plurality of tube outlets 62. Though positioning may occur in several positions, the heater 40 may be positioned in the manifold 60. Each tube outlet 62 is fluidly engaged to a flexible tube 64. The flexible tubes 64 each include a distal end 66, with respect to the manifold 60, that is extended through openings in the wall 44 surface such that the distal end 66 is extended into the enclosed wall space 42 of the wall 44. A plurality of tubes 64 is utilized so that air may be directed where needed and to ensure full coverage of all wall spaces 42 which may be separated from each other by interior structures, insulation and the like.

During usage, the blower 46 is turned on to supply pressurized air into the interior 14 of the compartment 12. The pressurized air may be heated such that is has an elevated temperature when it enters the tubes 58/64 and can thereafter be used for drying wetted interior surfaces of the walls 40. Intermittently, the control 38 will activate the atomizer 20 such that the fluid 24 is atomized and directed toward the flow of the pressurized air and is carried outwardly by the pressurized air through the air outlet 18 and into the air tubes 58/64. The atomized fluid moves through the interior spaces 42 of the wall 44. While traveling through the enclosed spaces, the atomized fluids trap particulate within the air and adhere to the surfaces bounding the enclosed spaces. While the atomizer 20 is being operated, the control 38 will typically turn off the heater 40 to prevent changing the state of the atomized fluid to a gas. Furthermore, the blower 46 will have a reduction in output to lower the back pressure created when the blower 46 is being operated at high output, such as when the assembly 10 is being used for drying alone.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A drying and airborne particle delivery system configured to be in fluid communication with an enclosed space of a wall, the system including:
a compartment having an interior, the compartment having an air inlet and an air outlet, wherein pressurized air enters into the interior through the air inlet and exits the interior through the air outlet, wherein pressurized air received by the air inlet is defined as received air;
an atomizer being in fluid communication with the interior and being configured to supply atomized fluid to the received air within the interior such that that the atomized fluid is carried outwardly through the air outlet; and
a control being in communication with the atomizer, the control turning on the atomizer intermittently for a preselected amount of time while the pressurized air flows through the compartment.

2. The drying and airborne particle delivery system according to claim 1, wherein a first condition is defined wherein the atomizer is turned off and no pressurized air flows through the interior, a second condition is defined wherein the pressurized air flows through the interior and the atomizer is turned off, a third condition is defined wherein the pressurized air flows through the interior and the atomizer is turned on, the second condition being maintained by the control for at least 50.0 minutes out of each hour that the pressurized air flows through the interior, the third condition being maintained by the control for at least 10.0 seconds out of each hour that the pressurized air flows through the interior.

3. The drying and airborne particle delivery system according to claim 1, wherein the atomizer comprises a sonic transducer configured to atomize a fluid in contact with the sonic transducer, the interior being configured to hold a quantity of fluid such that the sonic transducer is in contact with the fluid.

4. The drying and airborne particle delivery system according to claim 3, further including a reservoir configured to store the fluid, the reservoir being in fluid communication with the interior, a pump being in fluid communication with the container, the pump being configured to pump the fluid from the container into the interior when the pump is turned on.

5. The drying and airborne particle delivery system according to claim 2, wherein the atomizer comprises a sonic transducer configured to atomize a fluid in contact with the sonic transducer, the interior being configured to hold a quantity of fluid such that the sonic transducer is in contact with the fluid.

6. The drying and airborne particle delivery system according to claim 5, further including a reservoir configured to store the fluid, the reservoir being in fluid communication with the interior, a pump being in fluid communication with the container, the pump being configured to pump the fluid from the container into the interior when the pump is turned on.

7. The drying and airborne particle delivery system according to claim 1, wherein the atomizer comprises a nozzle directed into the interior.

8. The drying and airborne particle delivery system according to claim 7, further including reservoir configured to store a quantity of fluid, a conduit fluidly connecting the nozzle with the reservoir.

9. The drying and airborne particle delivery system according to claim 8, further including a pump being in fluid communication with the conduit, the pump being in communication with the control, wherein the pump pumps the fluid from the reservoir to the nozzle when the pump is turned on.

10. The drying and airborne particle delivery system according to claim 1, further including a heater being in fluid communication with the interior and being configured to heat the received air.

11. The drying and airborne particle delivery system according to claim 2, further including a heater being in fluid communication with the interior and being configured to heat the received air, the heater being in communication with the control, the control turning the heater only on during the second condition, the control turning the heater off during the third condition.

12. The drying and airborne particle delivery system according to claim 6, further including a heater being in fluid communication with the interior and being configured to heat the received air, the heater being in communication with the control, the control turning the heater only on during the second condition, the control turning the heater off during the third condition.

13. The drying and airborne particle delivery system according to claim 1, further including a blower being fluidly coupled to the air inlet and delivering the pressurized air to the air inlet when the blower is turned on.

14. The drying and airborne particle delivery system according to claim 12, further including a blower being fluidly coupled to the air inlet and delivering the pressurized air to the air inlet when the blower is turned on, the blower including at least a high output and a low output, the blower being in communication with the control, the control turning on the blower at the high output during the second condition, the control turning on the blower at the low output during the third condition.

15. The drying and airborne particle delivery system according to claim 2, further including a blower being fluidly coupled to the air inlet and delivering the pressurized air to the air inlet when the blower is turned on, the blower including at least a high output and a low output, the blower being in communication with the control, the control turning on the blower at the high output during the second condition, the control turning on the blower at the low output during the third condition.

16. The drying and airborne particle delivery system according to claim 1, wherein the control include a transceiver, the transceiver being configured to be in wireless communication with an actuator to allow remote actuation of the control.

17. A drying and airborne particle delivery system configured to be in fluid communication with an enclosed space of a wall, the system including:
a compartment having an interior, the compartment having an air inlet and an air outlet, wherein pressurized air enters into the interior through the air inlet and exits the interior through the air outlet, wherein pressurized air received by the air inlet is defined as received air;
an atomizer being in fluid communication with the interior and being configured to supply atomized fluid to the received air within the interior such that that the atomized fluid is carried outwardly through the air outlet;
a control being in communication with the atomizer, the control turning on the atomizer intermittently for a preselected amount of time while the pressurized air flows through the compartment, a first condition being defined wherein the atomizer is turned off and no pressurized air flows through the interior, a second condition being defined wherein the pressurized air flows through the interior and the atomizer is turned off, a third condition being defined wherein the pressurized air flows through the interior and the atomizer is turned on, the second condition being maintained by the control for at least 50.0 minutes out of each hour that the pressurized air flows through the interior, the third condition being maintained by the control for at least 10.0 seconds out of each hour that the pressurized air flows through the interior;
the atomizer comprising a sonic transducer configured to atomize a fluid in contact with the sonic transducer, the interior being configured to hold a quantity of fluid such that the sonic transducer is in contact with the fluid;
a heater being in fluid communication with the interior and being configured to heat the received air;
the heater being in communication with the control, the control turning the heater on during the second condition, the control turning the heater off during the third condition;
a blower being fluidly coupled to the air inlet and delivering the pressurized air to the air inlet when the blower is turned on, the blower including at least a high output and a low output, the blower being in communication with the control, the control turning on the blower at the high output during the second condition, the control turning on the blower at the low output during the third condition;
the control including a transceiver, the transceiver being configured to be in wireless communication with an actuator.
